(19) United States
(12) Patent Application Publication
(10) Pub. No.: US 2021/0236045 A1
(43) Pub. Date: Aug. 5, 2021

(54) DETECTION OF AN EPILEPTIC SEIZURE

(71) Applicant: LIVASSURED B.V., Vught (NL)

(72) Inventors: Theo Ary Asmund TIELENS, Vught (NL); Johannes STEENSMA, Vught (NL); Mark Auke Jan BLOEMENDAAL, Vught (NL)

(21) Appl. No.: 17/268,544

(22) PCT Filed: Aug. 27, 2018
(86) PCT No.: PCT/EP2018/073030

§ 371 Date: Feb. 15, 2021

(51) Int. Cl. A61B 5/00; A61B 5/11; A61B 5/0205; G16H 40/67

(52) U.S. Cl. CPC A61B 5/4094; A61B 5/6824; A61B 5/7282; A61B 5/1117; A61B 5/1121; A61B 5/02438; A61B 5/0205; A61B 5/7221; A61B 5/0004; G16H 40/67; A61B 2562/0219; A61B 5/6831

(57) ABSTRACT

The device includes a housing fastenable to a subject's upper arm wherein housing and upper arm have parallel longitudinal axes. The housing accommodates a control circuit including a processing unit, a physiological sensor and an acceleration sensor for providing a sensor signal and an acceleration signal, respectively to the processing unit. The processing unit includes an evaluation function for evaluating the sensor signal and providing a detection signal, a generator function generating a signal indicative of an occurrence of an epileptic seizure from said detection signal, a posture determination function using the acceleration signal and arranged within the housing for establishing an angle between the housing's longitudinal axis and a gravity vector, and for determining whether the angle is within a predetermined range corresponding to the upper arm lying down. The evaluation function further reduces a sensitivity of evaluating the sensor signal when the angle is within the predetermined range.

### FIELD OF THE INVENTION

The invention relates to a device for the detection of an epileptic seizure and a use of the device.

### BACKGROUND OF THE INVENTION

Sudden unexpected death of someone with epilepsy (SUDEP), may occur to a subject who was otherwise healthy. In SUDEP cases, no other cause of death is found when an autopsy is done. Each year, more than 1 out of 1,000 people with epilepsy die from SUDEP. If seizures are uncontrolled, the risk of SUDEP increases to more than 1 out of 150. These sudden deaths are rare in children but are known to be the leading cause of death in young adults with uncontrolled seizures.

The person with epilepsy is often found dead in bed and doesn't appear to have had a convulsive seizure. About a third of them do show evidence of a seizure close to the time of death. They are often found lying face down. No one is sure about the cause of death in SUDEP. Some researchers consider the possibility that a seizure may cause an irregular heart rhythm. More recent studies have suggested that the person may suffocate from impaired breathing, fluid in the lungs, and being face down on the bedding (source Epilepsy Foundation USA website).

Wearable sensor devices are known for monitoring physiological parameters such as heart rate, skin resistance, temperature, etc. Such devices can be worn on various body parts and limbs. Wearable sensor devices can for example be worn on the wrist. In other applications sensor devices may be worn on the upper arm.

Wearable sensor devices may have a physiological sensor such as a heart activity sensor which measures for example electrical heart activity, blood pressure variations or blood flow. An example of such a sensor is a photoplethysmographic sensor, which measures variations in light transmitted into a subject's skin. Light from underlying tissue is reflected to a photosensitive sensor. The amount of reflected light depends on blood pressure. While the blood pressure varies in time with the subject's heart rate, the amount of reflected light thus allows a heart rate to be established accurately.

The established heart rate can be used in epileptic seizure detection. The heart rate often shows variations typical for an epileptic seizure. When an epileptic seizure is detected, especially during sleep, care takers such as medical staff, nurses but also partners of patients and/or parents may be notified using alarm systems communicatively coupled to detection devices. Thus, SUDEP may be prevented when such a care taker is duly notified allowing intervention when required especially in the 20 minutes after the actual seizure (source Brian J Dlouhy, Brian K Gehlbach and George B Richerson, 2015, Journal of Neurology, Neurosurgery & Psychiatry 2016 87: 402-413).

Moreover, epileptic seizures may be detected using motion of the limbs.

Subjects suffering from epileptic seizures are actually most prone to SUDEP during the night lying in bed. Present epileptic seizure detecting devices suffer from false detections, related to postures deviating from lying down. In other circumstances however, detection may be required when the subject is incapacitated, and actually in an upright position not able to lie down.

### SUMMARY OF THE INVENTION

It is therefore an object of the invention to overcome the abovementioned problems and disadvantageous. The object is achieved in a device for the detection of an epileptic seizure. The device comprises a housing, wherein the housing has a baseplate and fastening means for fastening the housing to a subject's upper arm. The housing has a longitudinal axis parallel to the longitudinal axis of the upper arm.

The housing accommodates a control circuit, the control circuit comprises a processing unit, at least one physiological sensor connected to the processing unit, arranged for providing at least one sensor signal to the processing unit, an acceleration sensor connected to the processing unit, arranged for providing an acceleration signal to the processing unit.

The processing unit comprises at least one evaluation function corresponding to the at least one physiological sensor, wherein the at least one evaluation function is arranged for evaluating the sensor signal of the physiological sensor to provide a detection signal, and a generator function for generating a signal indicative of an occurrence of an epileptic seizure from said at least one detection signal, a posture determination function using the acceleration signal.

The processing unit further comprises a posture determination function using the acceleration signal, wherein the posture determination function is arranged within the housing for establishing an angle between said longitudinal axis of the housing and a gravity vector.

The processing unit further comprises a posture determination function, wherein the posture determination function is arranged for determining whether the angle is within a predetermined range corresponding to a lying down posture of the upper arm.

The at least one evaluation function is further arranged for reducing a sensitivity of the evaluating the sensor signal when the posture determination function determines that the angle from the posture determination function is within the predetermined range.

Subjects in an upright position do not likely suffer from epileptic seizure. By reducing sensitivity of the evaluation function for epileptic seizure when the subject wearing the device according to the invention is in an upright position, the detection becomes more selective for the kind of epileptic seizures which are known to be responsible for SUDEP, i.e. when the subject is asleep and thereby in a horizontal position.

In an embodiment, said at least one evaluation function comprises
establishing a parameter from the sensor signal;
establishing whether a value of said parameter complies with at least one predetermined parameter condition indicative of epileptic seizure, wherein said complying of said parameter value with at least one parameter value condition indicative of epileptic seizure comprises at least one of the parameter value being higher than or equal to a predetermined first threshold value, the parameter value being less than or equal to a predetermined second threshold value, and the parameter value lying in a predetermined range;
determining a duration time of said parameter value complying with the at least one predetermined parameter condition indicative of epileptic seizure;
generating the detection signal if the duration exceeds a time limit.

The detection of epileptic seizure may be performed using various parameters, such as heart rate and motion. The condition indicative of epileptic seizure however may be transient. To prevent false detection, the duration of the condition indicative of epileptic seizure indicative of can be determined, and when the duration is longer than a known predetermined time, epileptic seizure may be detected more selectively, i.e. with a lower chance of a false detection.

In an embodiment, the reducing the sensitivity of the evaluating the sensor signal comprises extending said time limit.

In an embodiment, the reducing the sensitivity of the evaluating the sensor signal comprises adapting said predetermined parameter condition indicative of epileptic seizure to more strict condition indicative of epileptic seizure. When the condition indicative of epileptic seizure indicative comprises a threshold which has to exceeded, the threshold may be raised. Otherwise, when the condition indicative of epileptic seizure indicative comprises another threshold which has to undershoot, the threshold may be lowered. When the condition indicative of epileptic seizure indicative comprises a parameter value range wherein the parameter is to be to detect, the range may be narrowed down.

In an embodiment, the reducing the sensitivity of the evaluating the sensor signal comprises cancelling said evaluating.

As an alternative, the sensitivity may be reduced to zero, by cancelling the evaluation. This may also be achieved by inhibiting the evaluation, or by suppression of the sensor signal.

In an embodiment, said at least one evaluation function comprises a heart activity evaluation function, wherein the heart activity evaluation function is arranged for evaluating a heart activity signal from a heart activity sensor and wherein the parameter comprises a heart rate.

Heart rate is a reliable parameter for detecting an epileptic seizure. The heart rate may for example be raised above a predetermined threshold value for a certain duration.

Alternatively, the heart rate may exhibit an increase above another threshold value in a certain time period to indicate an epileptic seizure.

In an embodiment, the heart activity sensor comprises a photoplethysmographic (PPG) sensor arranged in the baseplate to face the subject's upper arm. The PPG sensor may be advantageously positioned at the base plate of the device facing the subject's skin, to reliably measure variations of the subject's blood flow corresponding with the subject's heart rate, which can be determined from said variations.

This allows non-invasive measurement of the subject's heart rate. It eliminates the need for electrodes and thereby increases wearer's comfort and ease of use.

In an embodiment, said at least one evaluation function comprises a motion evaluation function, wherein the motion evaluation function is arranged for evaluating said acceleration signal, and wherein said parameter comprises motion frequency.

Motion of the limbs, especially of the arm whereupon the device is attached, provide another reliable parameter for detecting an epileptic seizure. Especially high frequency limb vibrations can be indicative for epileptic seizure when a duration of which exceeds a corresponding time limit. Such detection can be reliable also when the subject is in an upright position.

In an embodiment, the processing unit comprises a plurality of evaluation functions, and wherein the reducing the sensitivity is performed selectively on said plurality of evaluation functions depending on the angle.

This allows for specific situations to tune the evaluation to obtain a more selective detection wherein a specific evaluation for a sensor may be desensitized, i.e. reduced in sensitivity, and another evaluation is performed without desensitization the evaluation.

In an embodiment, at least one of said plurality of evaluation functions is arranged for reducing the sensitivity evaluating in another evaluation function, and wherein at least one other of said plurality of evaluation functions is arranged for being desensitized by yet another evaluation function.

This allows further enhancement of selectivity, wherein reduction of false detections in cases where specific combinations of evaluation functions represent mutually unlikely conditions indicative of epileptic seizure.

In an embodiment, said motion evaluation function is arranged for reducing the sensitivity said heart activity evaluation function.

This is an example of mutually unlikely conditions indicative of epileptic seizure.

In an embodiment, the fastening means are arranged for applying a lateral force to the housing for clamping the subject's upper arm. This ensures that the housing and thereby the acceleration sensor are aligned with the subject's upper arm on which the device is worn.

In an embodiment, the fastening means comprise a band. This allows the epileptic seizure detection device to be flexibly worn around a subject's limb, preferably the subject's upper arm.

In an embodiment, the band comprises an elastically extendible portion. This allows the epileptic seizure detection device to be worn comfortably and allows easy putting on and taking off.

In an embodiment, the predetermined range is from −45°-+45°.

This allows a broad range of postures to be assumed by the subject's limb wherein the generating of a signal indicative of an occurrence of an epileptic seizure is inhibited, thereby reducing the chance of false alarms.

In an embodiment, the device further comprises a wireless communication device connected to the processing unit, wherein the wireless communication device is arranged for communicating said signal indicative of an occurrence of an epileptic seizure.

This allows the signal indicative of an occurrence of an epileptic seizure to be issued, such that the subject or subject's care takers can take notice of the condition of the subject and act when necessary. The wireless communication device may for example facilitate wireless communication, whereby remote monitoring is achieved.

The object is further achieved in a use of a device for the detection of an epileptic seizure as described above, comprising:
attaching the device to an upper arm of a subject, wherein the baseplate of the device contacts the subject's upper arm such that the longitudinal axis of the housing is parallel to the longitudinal axis of the upper arm.

This allows reliable detection of the subject's posture, as it can be assumed that when the subject is lying down, the subject's upper arm has a horizontal position orientation.

### BRIEF DESCRIPTION OF THE DRAWINGS

Further features and advantages of the invention will become apparent from the description of the invention by way of exemplary and non-limiting embodiments of a device for the detection of an epileptic seizure according to the invention.

The person skilled in the art will appreciate that the described embodiments of the device for the detection of an epileptic seizure are exemplary in nature only and not to be construed as limiting the scope of protection in any way. The person skilled in the art will realize that alternatives and equivalent embodiments of the device for the detection of an epileptic seizure can be conceived and reduced to practice without departing from the scope of protection of the present invention.

Reference will be made to the figures on the accompanying drawing sheets. The figures are schematic in nature and therefore not necessarily drawn to scale. Furthermore, equal reference numerals denote equal or similar parts. On the attached drawing sheets:

FIG. 1a shows an isometric view of an epileptic seizure detection device according to an embodiment of the invention;

FIG. 1b shows a schematic representation of an epileptic seizure detection device according to an embodiment of the invention;

FIG. 2a shows a functional block diagram of an epileptic seizure detection device according to an embodiment of the invention;

FIG. 2b shows a detailed functional block diagram of an evaluation function of FIG. 2a;

FIG. 3a shows a subject in an upright position wearing an epileptic seizure detection device according to an embodiment of the invention;

FIG. 3b shows a vector diagram for the epileptic seizure detection device according FIG. 3a;

FIG. 4a shows a subject in a lying position wearing an epileptic seizure detection device according to an embodiment of the invention;

FIG. 4b shows a vector diagram for the epileptic seizure detection device according FIG. 4a.

### DETAILED DESCRIPTION OF EMBODIMENTS

Embodiments of the invention will be elucidated in the description below with reference to the drawings.

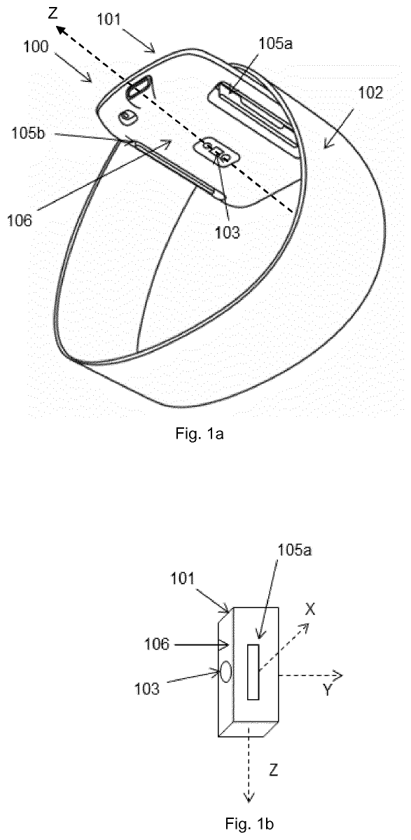

In FIG. 1 an epileptic seizure detection device 100 is shown having a housing 101, and a band 102 attached to the housing. A band 102 attached to the housing 101 allows the epileptic seizure detection device 100 to be worn preferably around an upper arm of a subject. The band 102 is preferably a band or strap, which is made of elastic material or which has at least an elastic portion. The housing 101 can be made of plastic. The housing 101 is provided with fasteners 105a, 105b for attaching the band 102 to the housing 101. A central axis Z of the housing 101 is shown by the dotted line.

The epileptic seizure detection device 100 is equipped with at least one physiological sensor 103. A preferred physiological sensor 103 is a heart activity sensor to which is further referred to in this text with reference numeral 103, however other physiological sensor types such as EEG electrodes, ECG electrode blood pressure, motion detectors, temperature, respiration, etc. may be used to establish respective physiological parameters which may be used to detect epileptic seizure.

The physiological sensor signal 208 may comprise a photoplethysmographic sensor which operates by sending light into the skin of the subject wearing the epileptic seizure detection device 100. is. The photoplethysmographic sensor in this example is located in a center region of the baseplate 106 of the housing 101. Any light reflected from within the skin of the subject, is detected by a photo detector comprised in the photoplethysmographic sensor. Variations in the detected reflected light provide a heart activity signal which allows a heartrate of the subject wearing the device to be established. The physiological sensor signal 208 may also be provided by means of an electrode pair for measuring heart activity signal electrically, i.e. electrocardiography. From the electrical heart activity signal a heart rate may be established.

The epileptic seizure detection device 100 is further provided with an acceleration sensor (not shown in FIG. 1a or FIG. 1b) accommodated within the housing 101, which provides an acceleration signal which allows a three-dimensional measurement of acceleration relative to a coordinate system of the sensor. By aligning the coordinate system of the acceleration sensor with the housing, an orientation of the housing central axis Z relative to gravity can be established. Within this application an orthogonal coordinate system for the acceleration sensor is assumed, but the skilled person will be able to apply other coordinate systems such as a spherical or non-orthogonal coordinate system as well. This also applies to the housing coordinate system.

In FIG. 1b, in a schematic overview, the coordinate system of the acceleration sensor, having x-y- and z directions, is shown relative to the device housing 101. The z-axis of the acceleration sensor is chosen to coincide with the central axis Z of the housing 101.

In use, the baseplate 106 can be pressed against the subject's upper arm. The fasteners 105a, 105b and the band 102 ensure that the central axis Z of the housing 101 with the coordinate system of the acceleration sensor is aligned parallel to the main longitudinal axis through the subject's upper arm. Simultaneously, the pressing against the subject's upper arm allow the physiological sensor signal 208 to contact the subject's upper arm skin and measure heart activity.

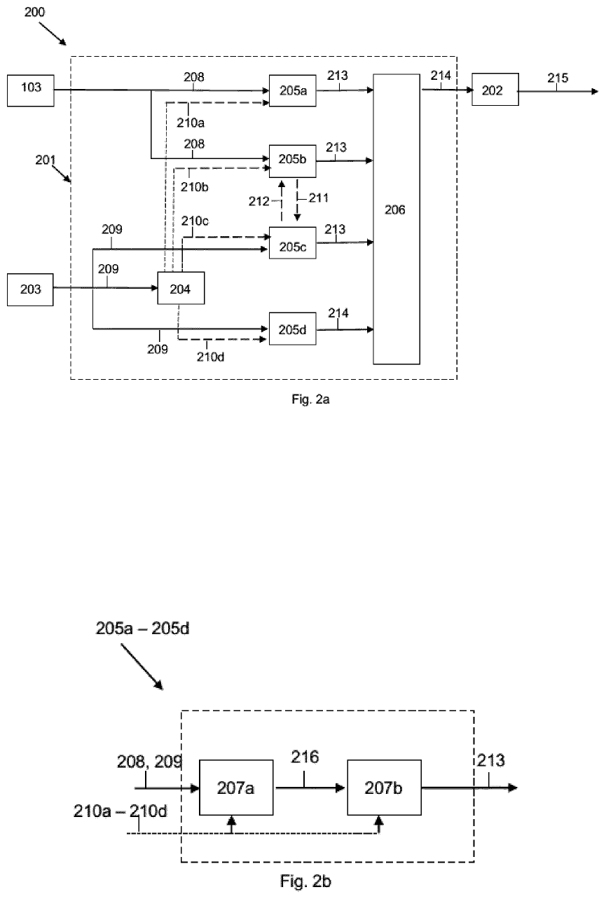

The epileptic seizure detection device 100 is provided within the housing 101 with a control circuit 200 as shown in FIG. 2a which comprise a processing unit 201 and electronic circuitry such as memory, and signal amplifiers, battery to operate the processing unit 201. The control circuit 200 interfaces the physiological sensor signal 208 and an acceleration sensor 203 to the processing unit 201. The processing unit 201 can be a microcontroller, microprocessor or any device arranged for executing program instructions stored in a program memory connected to or integrated with the processing unit 201.

The program instructions allow the processing unit 201 to capture and process the heart activity signal from the physiological sensor signal 208 and detect deviations which may be indicative for an epileptic seizure. Moreover, the program instructions allow the processing unit to capture the acceleration signal from the acceleration sensor to establish the subject's posture and to detect motion patterns which may also be indicative of an epileptic seizure. The subject's posture can be used to selectively use the heart activity signal and the acceleration signal to accurately and selectively determine occurrence of an epileptic seizure in different living situations of the subject.

In FIG. 2a, an exemplary block diagram of the control circuit 200 is shown having the processing unit 201 comprising circuitry for receiving or processing a physiological sensor signal 208 from the physiological sensor, i.e. heart activity sensor and an acceleration signal 209 from the acceleration sensor 203. The processing unit 201 performs the epileptic seizure detections and transmits a system detection signal 214 via a wireless communication device 202 as an alert transmission 215 to remote devices such as a remote alert apparatus or mobile phone for alerting care takers of a possible epileptic seizure. So, when a heart rate pattern is detected which corresponds to an epileptic seizure, an alert transmission 215 indicating this may be communicated to a remote signaling device via wireless communication device 202. Moreover, the signaling device may be equipped with at least one of a visual, audible and tactile alarm, allowing the care taker to be alerted of the possible epileptic seizure.

The wireless communication device 202 can for example operate in one of the ISM radio bands or in the DECT band. Communication may also be performed according to one or more of the 3G-5G, Wi-Fi, Bluetooth and DECT communication standards respectively.

In this example, the physiological sensor signal 208 is shown connected to the processing unit 201. The physiological sensor signal 208 is generated by the physiological sensor signal 208, which can be used in a plurality of evaluation functions 205a-205d. Likewise, the acceleration sensor 203 is connected to the processing unit 201. The acceleration signal 209 is generated by the acceleration sensor 203, which signal 209 may also be used in various evaluation functions 205a-205d.

As shown in FIG. 2a, the acceleration signal 209 is also used in a posture detection function 204, which allows the orientation of the acceleration sensor 203 to be determined relative to gravity, as will be further elaborated below.

Each of the evaluation functions 205a-205d are arranged to generate a detection signal 213 corresponding to an epileptic seizure. In generator function 206, the detection signals 213 from the various evaluation functions 205a-205d are logically combined using for example a logical OR-function, and a system detection signal 214 is generated, for communicating the likely detection of an epileptic seizure using wireless communication device 202.

The posture detection function 204 is arranged to determine the posture of the subject using the acceleration signal 209. Depending on the posture a desensitizing control signal 210a-210d can be generated which can be used by the respective evaluation functions 205a-205d for reducing sensitivity of the detection of epileptic seizure. The desensitizing control signal 210a-210d may indicate whether the posture of the subject is upright or lying down.

Moreover, it can be understood that the desensitizing control signals 210a-210d indicate a measure for upright position relative to a lying down position, which may control sensitivity of the respective evaluation functions. The closer the desensitizing control signal 210a-210d corresponds to an upright position, the more sensitivity reduction of the evaluation function 205a-205d is achieved.

Alternatively, the desensitizing control signals 210a-210d when indicating an upright position, may inhibit or suppress the evaluation function 205a-205d for detecting epileptic seizure.

In FIG. 2a is also shown that one of the evaluation functions 205a-205d may reduce sensitivity of another evaluation function. In this example is depicted a situation wherein evaluation function 205b reduces sensitivity of evaluation function 205c using control signal 211 and/or vice versa using control signal 212. For example, an evaluation function arranged for evaluating motion may reduce sensitivity another evaluation function arranged for evaluating heart rate.

In FIG. 2b, a block diagram of an evaluation function 205a-205d is shown in more detail. In parameter test function 207a a parameter can be established from the respective sensor signals 208, 209. The parameter can be compared to a predetermined threshold or a combination of predetermined thresholds to establish whether the parameter is above such threshold, undershoots such threshold or whether the parameter is within a predetermined range. When the parameter value exceeds or undershoots the thresholds or is out of range, primitive detection signal 216 is generated. The primitive detection signal 216 is then tested in timing function 207b for duration. When the duration of the primitive detection signal 216 exceeds or undershoots a predetermined duration or falls within a predetermined range of duration, the detection signal 213 is generated.

One of the evaluation functions 205a-205d may be arranged for evaluating a physiological sensor signal 208. In a first example in evaluation function 205a the physiological sensor signal 208 can be a heart activity sensor such as the already mentioned photoplethysmographic sensor, which can be used for determination of an average heart rate parameter, which can be compared with an average heart rate threshold. When the average heart rate parameter value is above a certain percentage of an average heart rate threshold value, parameter test function 207a generates primitive detection signal 216. Detection of a possible epileptic seizure is performed when timing function 207b determines that the duration of the primitive detection signal persists longer than or equal to an average heart rate duration value. In this case the detection signal 213 is generated.

In a second example in the evaluation functions 205b the physiological sensor signal 208 cab be a heart activity sensor such as the already mentioned photoplethysmographic sensor, which can be used for determination of a heart rate percental increase in predetermined time intervals. When parameter test function 207a establishes that the percental heart rate difference relative to a base heart rate is above a percental heart rate increase threshold value, primitive detection signal 216 is generated. In timing function 207b, it is then determined whether this primitive detection signal 216 indicative of a possible epileptic seizure is achieved within a predetermined relative heart rate increase interval. If so, the timing function 207b, and therewith evaluation function 205a-205d, will generate detection signal 213.

In a third example, the evaluation function 205c can be arranged as motion or vibration evaluation functions wherein the acceleration signal 209 is used. In the evaluation function 205c, the parameter test function 207a can use the acceleration signal 209 to establish a motion frequency parameter of the epileptic seizure detection device 100. When the motion frequency parameter value is in a motion frequency band, the primitive detection signal 216 is generated.

When the primitive detection signal 216 persists with or without short interruptions longer than a first predetermined motion frequency duration, detection signal 213 is generated by timing function 207b.

In a fourth example, in evaluation function 205d, the parameter test function 207a can use the acceleration signal 209 to establish a motion frequency parameter of the epileptic seizure detection device 100. When the motion frequency parameter value is in said motion frequency band, the primitive detection signal 216 is generated.

When the primitive detection signal 216 persists uninterruptedly longer than a second predetermined motion frequency duration, which is shorter than the first predetermined motion frequency duration, detection signal 213 is generated by timing function 207b.

In a fifth example another evaluation function can be arranged as motion evaluation function, the parameter test function 207a can use the acceleration signal 209 to establish a motion amplitude parameter of the epileptic seizure detection device 100. When the motion amplitude parameter value exceeds a motion amplitude threshold value, the primitive detection signal 216 is generated.

When the primitive detection signal 216 persists longer than a predetermined motion frequency duration, detection signal 213 is generated by timing function 207b.

The reducing sensitivity of the evaluation 205a-205d of the signal the heart activity sensor signal 208 or the acceleration sensor signal 209 the detection signals 213 and system detection signal 214 indicative of an occurrence of an epileptic seizure can be performed by the program instructions of the processing unit 201.

The various respective thresholds in the parameter test function 207a of the evaluation functions 205a-205d can be adjusted depending on the respective desensitizing control signals 210a-210d to more restrictive values. When for example the parameter is to exceed a threshold to generate the primitive detection signal 216, the threshold may be set to a higher level. Otherwise, when for example the parameter is to undershoot a threshold to generate the primitive detection signal 216, the threshold may be set to a lower level. Otherwise, when for example the parameter is to fall in a range to generate the primitive detection signal 216, the range limits may be set to narrower range.

Likewise, in the timing function 207b, the predetermined duration by which timing function 207b evaluates the primitive detection signal 216 may be set to a longer duration or a shorter duration or a narrower timing interval depending on the required detection.

In the examples given above, the evaluation functions 205a-205d may be selectively reduced in sensitivity. It is for example possible to reduce sensitivity of evaluation functions 205a and 205d using desensitizing control signals 210a and 210d to allow the epileptic seizure detection device 100 to respond to short term heart rate increase or long-term motion or vibration. This allows performing epileptic seizure detections in specific situations, wherein the subject is not explicitly lying down. In the given example, detection is still possible when the subject is for example trapped in a small space such as a lavatory and undergoing an epileptic seizure.

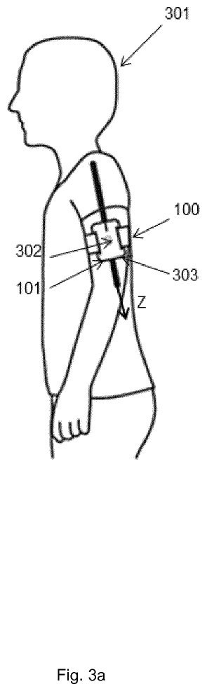

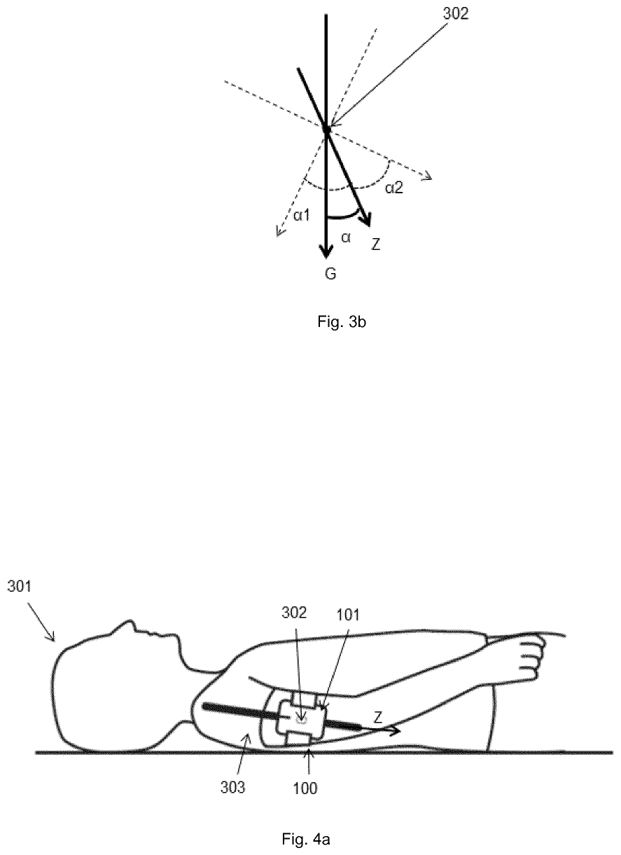

In FIG. 3a a subject 301 is shown wearing the epileptic seizure detection device 100 around the upper arm 303. The upper arm 303 is the preferred limb for detecting un upright position of the subject. The processing unit 201 is arranged such that the epileptic seizure detection sensitivity is reduced when the subject, i.e. the subject's upper arm 303 is in an upright position.

The upright position in FIG. 3a is shown by an axis Z through the subject's upper arm 303. The device 100 has an acceleration sensor reference point 302 for the acceleration sensor 203. In FIG. 3b, a corresponding z-axis Z is shown in a vector diagram having an angle α relative to the gravity vector G detected by the acceleration sensor 203. When the angle α between the z-axis Z and gravity vector G is between the boundaries indicated by α1 and α2, it is a shown that the device housing 101 is in an upright position, and thereby that the upper arm 303 of the subject is in an upright position and that the subject 301 is in an upright position. This condition is determined by posture evaluation function 204 as shown in FIG. 2a.

The acceleration sensor 203 is arranged for measuring acceleration components relative to an acceleration sensor's x-axis, y-axis and z-axis. Since the acceleration sensor 203 is accommodated in the housing, the acceleration sensor's x-axis, y-axis and z-axis have a fixed relationship with the housing x-axis X, y-axis Y, and z-axis Z. For the sake of simplicity, it is assumed that the acceleration sensor's x-axis, y-axis and z-axis are the same as the housing x-axis, y-axis and z-axis as shown in FIGS. 1a and 3. A different relationship between the sensor's xyz-axes and the housing xyz-axes may be contemplated without leading away from the inventive concept of the invention.

The angle α is determined from the absolute value of the component of gravity vector G relative to the acceleration sensor 203 z-axis Z. This can be performed by the posture detection function 204 calculating the formula [1]:

$$∝ = arccos [ \sqrt{\frac{{z}^{2}}{{x}^{2} + {y}^{2} + {z}^{2}}} ] , \qquad [ 1 ]$$

wherein z represents the component of the gravity vector parallel to the z-axis determined by the acceleration sensor, wherein x represents the component of the gravity vector relative to the x-axis determined by the acceleration sensor, and wherein y represents the component of the gravity vector relative to the y-axis determined by the acceleration sensor 203.

By taking the absolute value of the z-component, i.e. the z-component may be positive or negative, the epileptic seizure detection device 100 can be worn attached to the subject's upper arm in an upright position, or upside down.

The skilled person will understand that the angle α can be determined using alternative calculation methods. For example, the angle can be determined from the arctangent of the gravity vector in the x-y plane and the absolute z-component. The skilled person will further understand that other trigonometric determinations can be made which are equivalent for determining an angle α.

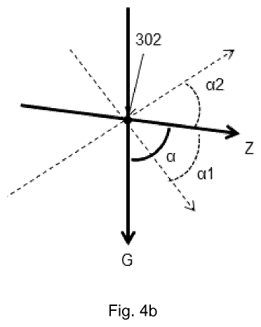

In FIG. 4a, the subject 301 is shown lying down. In FIG. 4b, the corresponding gravity vector G is shown relative to the z-axis of the acceleration sensor. The angle α between the z-axis and the gravity vector G can be wider than shown in FIG. 3b. In fact, the angle α between the gravity vector G and the Z-axis through the acceleration sensor reference point 302 of the device housing 101 as shown in FIG. 4b exhibits an angle α which lies beyond the range α1, α2. Beyond this range, the sensitivity of epileptic seizure evaluation of the processing unit 201 is not reduced.

Values for α1, α2 may for example be chosen in the order of −45° and +45°, however other or smaller ranges within this range may be contemplated.

It will be clear to a person skilled in the art that the scope of the present invention is not limited to the examples discussed in the foregoing but that several amendments and modifications thereof are possible without deviating from the scope of the present invention as defined by the attached claims. While the present invention has been illustrated and described in detail in the figures and the description, such illustration and description are to be considered illustrative or exemplary only, and not restrictive.

The present invention is not limited to the disclosed embodiments. Variations to the disclosed embodiments can be understood and effected by a person skilled in the art in practicing the claimed invention, from a study of the figures, the description and the attached claims. In the claims, the word “comprising” does not exclude other steps or elements, and the indefinite article “a” or “an” does not exclude a plurality. The mere fact that certain measures are recited in mutually different dependent claims does not indicate that a combination of these measures cannot be used to advantage. Any reference numerals in the claims should not be construed as limiting the scope of the present invention.

REFERENCE NUMERALS

| | |
|---|---|
| 100 | epileptic seizure detection device |
| 101 | housing |
| 102 | band |
| 103 | heart activity sensor |
| 105a, 105b | fasteners |
| 106 | base plate |
| 200 | control circuit |
| 201 | processing unit |
| 202 | wireless communication device |
| 203 | acceleration sensor |
| 204 | posture detection function |
| 205a-205d | evaluation function |
| 206 | generator function |
| 207a | parameter test function |
| 207b | timing function |
| 208 | physiological sensor signal |
| 209 | acceleration sensor signal |
| 210a-210d | desensitizing control signal |
| 211, 212 | mutual desensitizing control signal |
| 213 | detection signal |
| 214 | system detection signal |
| 215 | alert transmission |
| 216 | primitive detection signal |
| 301 | subject |
| 302 | acceleration sensor reference point |
| 303 | subject's upper arm |
| G | gravity vector |
| X | x-axis |
| Y | y-axis |
| Z | z-axis |

What is claimed is:

1. A device for the detection of an epileptic seizure, comprising:
a housing, the housing having a baseplate and fastening means for fastening the housing to a subject's upper arm having a longitudinal axis of the housing parallel to the longitudinal axis of the upper arm; wherein
the housing accommodates a control circuit, the control circuit comprising
a processing unit;
at least one physiological sensor connected to the processing unit, arranged for providing at least one sensor signal to the processing unit;
an acceleration sensor connected to the processing unit, arranged for providing an acceleration signal to the processing unit;
wherein
the processing unit comprises
at least one evaluation function each of which corresponding to one of the at least one physiological sensor wherein the evaluation function is arranged for evaluating the sensor signal of the at least one physiological sensor to provide a detection signal; and
a generator function for generating a signal indicative of an occurrence of an epileptic seizure from said at least one detection signal;
a posture determination function using the acceleration signal
wherein the posture determination function is arranged within the housing for establishing an angle (α) between said longitudinal axis of the housing and a gravity vector; and
wherein the posture determination function is further arranged for determining whether the angle (α) is within a predetermined range (α1, α2) corresponding to a lying down posture of the arm; and
wherein the at least one evaluation function is further arranged for reducing a sensitivity of the evaluating the physiological sensor signal when the posture determination function determines that the angle (α) from the posture determination function is within the predetermined range (α1, α2).

2. The device according to claim 1, wherein said at least one evaluation function comprises
establishing a parameter from the sensor signal;
establishing whether a value of said parameter complies with at least one predetermined parameter condition indicative of epileptic seizure, wherein said complying of said parameter value with at least one parameter value condition indicative of epileptic seizure comprises at least one of the parameter value being higher than or equal to a predetermined first threshold value, the parameter value being less than or equal to a predetermined second threshold value, and the parameter value lying in a predetermined range;
determining a duration time of said parameter value complying with the at least one predetermined parameter condition indicative of epileptic seizure;
generating the detection signal if the duration exceeds a time limit.

3. The device according to claim 2, wherein the reducing the sensitivity of the evaluating the sensor signal comprises extending said time limit.

4. The device according to claim 2, wherein the reducing the sensitivity of the evaluating the sensor signal comprises adapting said predetermined parameter condition indicative of epileptic seizure indicative of epileptic seizure to more strict condition indicative of epileptic seizure.

5. The device according to claim 2, wherein the reducing the sensitivity of the evaluating the sensor signal comprises cancelling said evaluating.

6. The device according to claim 3, wherein said at least one evaluation function comprises a heart activity evaluation function, wherein the heart activity evaluation function is arranged for evaluating a heart activity signal from a heart activity sensor and wherein the parameter comprises a heart rate.

7. The device according to claim 5, wherein the physiological sensor comprises a photoplethysmographic sensor arranged in the baseplate to face the subject's upper arm.

8. The device according to claim 3, wherein said at least one evaluation function comprises a motion evaluation function, wherein the motion evaluation function is arranged for evaluating said acceleration signal, and wherein said parameter comprises motion frequency.

9. The device according to claim 8, wherein the processing unit comprises a plurality of evaluation functions, and wherein the reducing the sensitivity is performed selectively on said plurality of evaluation functions depending on the angle (α).

10. The device according to claim 9, wherein at least one of said plurality of evaluation functions is arranged for reducing the sensitivity evaluating in another evaluation function, and wherein at least one other of said plurality of evaluation functions is arranged for being desensitized by yet another evaluation function.

11. The device according to claim 10, wherein said motion evaluation function is arranged for reducing the sensitivity said heart activity evaluation function.

12. The device according to claim 1, wherein the predetermined range (α1, α2) for the angle (α) is −45°-+45°.

13. The device according to claim 1, further comprising a wireless communication device connected to the processing unit, wherein the wireless communication device is arranged for communicating said signal indicative of an occurrence of an epileptic seizure.

14. A method for the detection of an epileptic seizure using the device according to claim 1, comprising:
obtaining the device of claim 1 and
attaching the device to an upper arm of a subject, wherein the baseplate of the device contacts the subject's upper arm such that the longitudinal axis of the housing is parallel to the longitudinal axis of the upper arm.

15. The device according to claim 4, wherein said at least one evaluation function comprises a heart activity evaluation function, wherein the heart activity evaluation function is arranged for evaluating a heart activity signal from a heart activity sensor and wherein the parameter comprises a heart rate.

16. The device according to claim 5, wherein said at least one evaluation function comprises a heart activity evaluation function, wherein the heart activity evaluation function is arranged for evaluating a heart activity signal from a heart activity sensor and wherein the parameter comprises a heart rate.

17. The device according to claim 4, wherein said at least one evaluation function comprises a motion evaluation function, wherein the motion evaluation function is arranged for evaluating said acceleration signal, and wherein said parameter comprises motion frequency.

18. The device according to claim 5, wherein said at least one evaluation function comprises a motion evaluation function, wherein the motion evaluation function is arranged for evaluating said acceleration signal, and wherein said parameter comprises motion frequency.

19. The device according to claim 2, wherein the predetermined range (α1, α2) for the angle (α) is −45°-+45°.

20. The device according to claim 2, further comprising a wireless communication device connected to the processing unit, wherein the wireless communication device is arranged for communicating said signal indicative of an occurrence of an epileptic seizure.
